(19) United States
(12) Patent Application Publication
(10) Pub. No.: US 2026/0092027 A1
(43) Pub. Date: Apr. 2, 2026

(54) HETEROGENEOUS CATALYST COMPRISING PLATINUM AND RUTHENIUM ON A ZEOLITE CARRIER AND THE USE OF SUCH CATALYST IN A PROCESS FOR PRODUCING AN ETHER

(71) Applicants: Dow Global Technologies LLC, Midland, MI (US); The Board of Trustees of the University of Illinois, Urbana, IL (US)

(72) Inventors: Jing Luo, Midland, MI (US); Xue Chen, Manvel, TX (US); David G. Barton, Midland, MI (US); David W. Flaherty, Urbana, IL (US); Claudia Eugenia Berdugo-diaz, Urbana, IL (US); Yangsik Yun, Champaign, IL (US); Jieun Lee, Urbana, IL (US)

(21) Appl. No.: 19/111,526

(22) PCT Filed: Oct. 11, 2023
(86) PCT No.: PCT/US2023/076537

§ 371 Date: Mar. 13, 2025

Related U.S. Application Data
(60) Provisional application No. 63/420,983, filed on Oct. 31, 2022.

(51) Int. Cl. C07C 41/20; B01J 29/22; C07C 43/04; C07C 43/06

(52) U.S. Cl. CPC C07C 41/20; B01J 29/22; C07C 43/04; C07C 43/06

(57) ABSTRACT

A process for producing an ether including treating (a) an ester with (b) hydrogen in the presence of (c) a heterogeneous catalyst to reduce the ester by hydrogenation to form an ether product, wherein the heterogeneous catalyst comprises platinum and ruthenium deposited on a zeolite support.

### FIELD

The present invention relates to processes for producing an ether compound; and more specifically, the present invention relates to processes for producing an ether compound directly from an alkyl ester using molecular hydrogen on a heterogeneous catalyst.

### BACKGROUND

Ethers are used in various applications, including as a solvent. Ethers are particularly desirable for use as a solvent in applications because ethers possess excellent solvency, chemical stability and compatibility with other organic solvents and formulated products. Known routes of synthesizing ethers include the following three routes: (1) alkyl halides treated with alkoxides (so called the “Williamson ether synthesis”); (2) alcohol addition to an olefin; and (3) acid catalyzed coupling of alcohols. However, the above three routes have undesirable limitations including: (1) use of strongly acidic or basic conditions which can lead to competing elimination reactions that produce undesired olefins; (2) limited options of bio-sourced raw materials due to lack of reactivity with the above reactions which limit the structural variety of products; and (3) use of toxic raw materials and generation of waste streams in a manufacturing process. Therefore, what is desired is to provide a viable route for producing an ether that can be successfully scaled up commercially without the limitations of the above known routes.

For example, heretofore the known methods for producing an ether include the following: (1) a process using metal hydride/Lewis acid complexes, or hydrosilanes as stoichiometric hydride donors with precious metal catalysts as disclosed in J. Org. Chem., 2007, 72, 5920-5922; Tetrahedron Letters, 2017, 58, 3024-3027; (2) a process for producing a thionate (a salt or ester of thionic acid) such as a thioether (a sulfide which is a bonded compound of sulfur and two organic residues) as disclosed in J. Org. Chem., 1981, 46, 831-832; (3) a process for the catalytic reduction of α-monoglycerides with a 5 percent (%) Pd/C catalyst mixed with an acid cocatalyst at about 700 psi (4.8 megapascals [MPa]) and 120 degrees Celsius (° C.) as disclosed in U.S. Pat. No. 8,912,365; (4) a non-direct process for the hydrogenation of ethyl acetate into ethanol intermediates that subsequently couple to form a symmetrical ether by-product on Re/(gamma-Al₂O₃) or Re/(theta-Al₂O₃) at up to 4.6% conversion and 57% selectivity as disclosed in Russian Chemical Bulletin 1988, 37 (1), 15-19 and Russian Chemical Bulletin 1986, 35, 280-283; and (5) a process for the hydrogenation of lactone to cyclic ethers, for example (e.g.), for production of tetrahydrofuran, in high selectivities such as greater than (>) 90% using various metal catalysts on various support carriers as disclosed in U.S. Pat. Nos. 3,370,067; 3,894,054; and 4,973,717. (6) a process using homogeneous metal complex catalysts (e.g., a ruthenium/triphos complex) which require impractical separation of catalyst from product. Angewandte Chemie, International Edition, 2015, 54, 5196-5200; ChemSusChem, 2016, 9, 1442-1448. (7) a process using Co/γ-Al2O3 to hydrogenate ethyl butyrate to products including ethanol, butanol, ethoxy butane, butyl butyrate, and C1-C4 linear alkanes. The unsymmetric ether, ethyl butyl ether, has selectivities lower than 9%. Journal of Catalysis, 2011, 277, 27-35. (8) a process using ZrO₂ supported Pt—Mo catalyst to selectively hydrogenate esters to unsymmetrical ethers in high yield, for example, 72% yield of butyl ethyl ether from ethyl butyrate on Pt—Mo/ZrO₂. JACS Au, 2022, 2 (3), 665-672. The catalysts reported in this publication include Pt—Mo/ZrO₂, Ru—Mo/ZrO₂, Rh—Mo/ZrO₂, Pd—Mo/ZrO₂, Pt—Re/ZrO₂, Pt—W/ZrO₂, Pt—V/ZrO₂, Pt—Mo/TiO₂, Pt—Mo/hydroxyapatite, Pt—Mo/CeO₂, Pt—Mo/MgO, Pt/MoO₃, Pt/ZrO₂, Mo/ZrO₂. The ester substrates investigated includes but are not limited in isobutyl acetate, 2-methyl butyrate, alkyl benzoates, isopropyl cyclohexane carboxylate, glyceryl ether, etc. However, the results reported in this paper cannot be reproduced in this work. Only less than 12% unsymmetrical ether selectivity was obtained under similar conditions on Pt—Mo/ZrO₂ catalyst. Comparative examples of such catalyst are included in this invention.

In a recently filed patent application, U.S. 63/107,739, a process to directly convert ester to ether with transition metals supported by metal oxide supports, such Nb₂O₅ and WO₃ is described. The invention reported high direct selectivity of ether formation via hydrogenation, however, the ether product absolute selectivities from hydrogenation were generally low, typically in a range of 5 to 10% with a maximum value of 16%. Further improvement of the catalyst selectivity is required in order to make the process economically valuable.

In another recently filed patent application, U.S. 63/276,308, a process for producing an ether from an ester with hydrogen molecule over heterogeneous catalyst comprising sulfonic acid functionalized SiO₂. The absolute ether product selectivity achieved from this class of catalysts is reported between 20˜35%.

In another recently filed patent application U.S. 63/276,311 a class of transition metal on zeolite support catalysts was described, which achieved ether product absolute selectivities between about 20 to 50%.

It would therefore be desirable to have improved catalysts for directly producing ethers from esters that can be commercially manufactured and that provide advantages over existing processes, including increased hydrogenation rates and improved absolute and/or direct selectivity.

### SUMMARY

The present invention is directed to new processes for producing an ether product from an ester starting raw material using bimetallic transition metal catalysts on zeolite carriers.

In a broad embodiment, a process of the present invention includes producing an ether by hydrogenation of an ester in the presence of a heterogeneous catalyst.

In one embodiment, a process of the present invention includes direct selective reduction of carboxylic acid derivatives into ethers using molecular hydrogen and a proper catalyst formulation for achieving a high (e.g., >10%) absolute ether selectivity with a high (e.g., >80%) direct ether selectivity. Absolute ether selectivity is the percentage of the total products formed in the reaction, while the direct ether product selectivity is the percentage of direct ether product over the total ether products.

In another embodiment, a process of the present invention for producing an ether comprises mixing: (a) at least one ester with (b) hydrogen in the presence of (c) a heterogeneous bimetallic catalyst to reduce the ester by hydrogenation to form an ether.

In still another embodiment, the present invention includes a solvent comprising the above ether product produced by the above process.

Some of the advantageous features that can be provided by one or more embodiments of the process of the present invention include, for example:

(1) An active catalyst is used in a one-step process. The catalyst is active for direct hydrogenation of an ester to reduce the ester to form an ether, rather than going through a known two-step ether formation process such as (i) ester hydrogenolysis to form alcohol followed by (ii) alcohol dehydration.

(2) A relatively inexpensive route is used. The process employs inexpensive molecular hydrogen as a reduction agent, rather than employing expensive hydrosilanes, metal hydrides, or metal hydride/Lewis acid complexes as a hydride donor. The highly reactive hydrosilanes or metal hydride employed in prior art processes also requires the design of a complicated and expensive process to ensure the safety of the operators running the prior art processes.

(3) A bimetallic heterogeneous catalyst is used. The catalyst being heterogeneous rather than homogeneous, can contribute to lower manufacturing costs due to catalyst recyclability and separation.

(4) An efficient process is used.

(5) A flexible process is used. The process is applicable to a general ester compound (either cyclic or acyclic) as a feed material; and the process is not limited to a specified ester compound.

### DETAILED DESCRIPTION

In one embodiment, the present invention includes a distinct and novel method for synthesizing ethers from esters with platinum-ruthenium bimetallic heterogeneous catalysts. The reactions of ester on heterogeneous catalysts include various chemical reaction routes or pathways, for example, hydrogenolysis, hydrolysis, dehydration, hydrogenation, and transesterification. In a general embodiment, the process of the present invention includes producing an ether by hydrogenation of an ester, such as a propyl acetate, in the presence of a heterogeneous catalyst. The present invention's novel hydrogenation reaction pathway or scheme, for example, the hydrogenation of propyl acetate reduction reaction scheme with R₁ being —CH₃ and R₂ being —CH₂CH₃, is generally illustrated as Reaction Scheme (I) as follows:

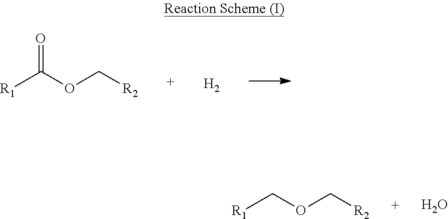

In the above Reaction Scheme (I), water is generated by the reduction process; and the generated water can be separated by conventional processes such as distillation or other procedures known in the art. Functional groups R₁ and R₂ can be alkyl functional groups including straight or branched-chain alkyl groups, cyclic or non-cyclic alkyl groups; and mixtures thereof. Examples of the esters herein include but are not limited to ethyl acetate, propyl acetate, butyl acetate, ethyl propionate, butyl propionate and mixtures thereof. The desired ether product resulting from the above Reaction Scheme (I) can be a symmetric ether when R₁ is equivalent to R₂; or an unsymmetric ether when R₁ is not equivalent to R₂, for example, the unsymmetric ether can be ethyl propyl ether.

A “symmetric ether” herein means an ether that contains two identical functional groups, wherein R₁ is identical to R₂. An “unsymmetric ether” herein means an ether that contains two different functional groups, where R₁ is not identical to R₂.

In the present invention the desired reaction scheme, Reaction Scheme (I), is a direct hydrogenation route to obtain the desired ether product. By “direct hydrogenation” or “direct selective reduction” it is meant that carbonyl oxygen is removed from ester (R₁COOCH₂R₂) by hydrogenation to form ether (R₁CH₂OCH₂R₂) while maintaining the alkoxyl group intact. The present invention process is different from previously known processes because the present invention process does not undergo a typical route for ester (R₁COOCH₂R₂) hydrogenation, where ester first breaks into two alcohols (R₁CH₂OH+R₂CH₂OH molecules via hydrogenolysis and then subsequently forms a mixture of ethers (R₁CH₂OCH₂R₁+R₁CH₂OCH₂R₂+R₂CH₂OCH₂R₂) via dehydration. Direct hydrogenation can maintain the structure of the ether from ester by only eliminating the carbonyl oxygen. Thus, an unsymmetric ester provided to this process advantageously results in the direct production of an unsymmetric ether because this process does not break the ester into two alcohol molecules via hydrogenolysis. The selectivity of ethyl propyl ether in the reaction examples are listed in Table III and Table V described below in the Examples.

The term “direct ether product” herein means an ether that is formed by a one-step reduction process of ether from ester.

The term “indirect ether product” herein means an ether that is formed by a two-step reduction process of ether from ester including the steps of: (i) hydrogenolysis and (ii) dehydration.

The term “direct ether product selectivity” herein means the percentage of the direct ether product over the total ether products. For example, for the reduction of propyl acetate, ethyl propyl ether is the direct ether product, and the direct ether product selectivity is the percentage of ethyl propyl ether over the total ether products (ethyl propyl ether+dipropyl ether+diethyl ether).

The term “ether product absolute selectivity” herein means the percentage of the ether product in the total products formed in the reaction (e.g., ethers, alcohols, and alkanes).

Advantageously, one unique factor of the present invention includes an increase in direct ether selectivity using the one-step process of the present invention versus the known two-step process.

In one embodiment, a process of the present invention for producing an ether comprises treating (a) an ester with (b) hydrogen in the presence of (c) a heterogeneous catalyst to reduce the ester by hydrogenation to form an ether.

In one desirable embodiment and as shown in Reaction Scheme (I) above, a process of the present invention for producing an ether comprises the steps of: (A) feeding into a reactor, an ester compound, component (a), such as propyl acetate; (B) feeding into the reactor, hydrogen, component (b), to form a hydrogen atmosphere in the reactor; and (C) charging the reactor with a heterogeneous catalyst system, component (c), comprising a platinum-ruthenium bimetallic transition metal on a zeolite support; sufficient to generate a hydrogenation reaction in the reactor; and (D) heating the contents of the reactor, components (a)-(c), at a temperature sufficient to reduce the ester compound to form an ether compound. For example, the heating step (D), can take place at a temperature of from 350 Kelvin (K) to 650 K. It will also be readily understood by those in the art that the order of the recited steps can be altered in certain circumstances, or steps may be done simultaneously

As is generally known in the art a “heterogeneous catalyst” refers to a catalyst whose phase (e.g., solid, liquid or gas) is different from the phase of the reactants. For example, the reactants (esters and hydrogen) and product (e.g., ether) can be in the liquid or gas phase while the catalyst is a solid.

In the present invention, the heterogeneous bimetallic catalytic system of the present invention is a combination of platinum-ruthenium metals and a zeolite support. For instance, the catalyst used in some embodiments of the present invention can include from 0.1 weight percent (wt %) to 20 wt % of the transition metals supported on zeolite support member. Preferably, the molar ratio of platinum to ruthenium is from 0.1 to 10, more preferably from 0.5 to 5, with rations of about 1:1 (such as (0.9 to 1.1) being most preferred.

As is generally known in the art, zeolites are crystalline aluminosilicate materials composed of corner-sharing AlO₄ and SiO₄ tetrahedra joined into three-dimensional frameworks having pores of molecular dimensions. The presence of aluminum in the zeolite framework results in a negative charge that is balanced by cations. The zeolites in this invention have a framework type which may advantageously be selected from the group consisting of the following framework types FAU, MOR, BEA, CHA, FER, MFI, and combinations thereof, the framework types corresponding to the naming convention of the International Zeolite Association. The preferred Zeolite framework type for the present invention is MOR.

Atomic Si/Al ratios are often used in the practice of catalysis to characterize the zeolites and refers the number of silicon containing sites (typically SiO₂) present per alumina containing site (typically HAlO₂). The zeolites for use in the present invention can have an Si:Al ratio in the range of from 1 to 300. In some embodiments the ratio may be 5 to 250. Si:Al ratios in the range of from 75:125 may be preferred for some applications of this invention.

The synergistic effect from both the bimetallic transition metal component and the zeolite support component promotes the direct ether reduction route, as described in the reaction pathway Reaction Scheme (I). Without the combination of a transition metal and a zeolite support member as disclosed herein, an undesirable two-step ether formation route would take place. Steady-state rates and product selectivity for competing reaction pathways for a model ester compound are obtained in, for example, a packed-bed reactor and/or a trickle bed reactor as functions of reactant pressures, temperature, and ester conversion, which are controlled via the surface residence time.

The ester compound, component (a), to be reduced to an ether, can include one or more ester compounds, including, for example, a carboxylic acid derivative; an ester containing straight or branched-chain alkyl groups, and cyclic or non-cyclic alkyl groups; and mixtures thereof. In some embodiments, an unsymmetric ether includes a Reaction Scheme (I) wherein the R₁ groups are not equal to the R₂ groups. In some embodiments, the ester useful in the present invention can be, for example, propyl acetate (available from Sigma-Aldrich); butyl acetate (available from Sigma-Aldrich); butyl propionate, glycerin ester; and mixtures thereof.

The concentration of the ester, component (a), is not particularly critical. However, it may be advantageous, in some embodiments, for the ester to be present in an amount of at least 1 wt % so as to provide a desirable production rate and/or avoid an increase in separation costs. In some embodiments, the concentration of the ester is from 1 wt % to 100 wt %. The concentration of the ester is based on the total weight of ester compounds in the liquid feed raw material.

The concentration of the hydrogen, component (b), useful in the process of the present invention includes, for example, from 3 wt % to 100 wt % in one embodiment, from 10 wt % to 100 wt % in another embodiment, and from 50 wt % to 100 wt % in still another embodiment. Hydrogen with a low concentration of, for example less than (<) 3 wt % may decrease the reactivities or ether selectivities; and therefore, in such a case an undesirable increase of the reaction pressure would be required. The concentration of the hydrogen is based on the total weight of hydrogen in the gas feed raw material.

In a broad embodiment, the catalyst used in the process, component (c), of the present invention, can include one or more heterogeneous catalyst compounds. The catalysts used in the present invention process include, for example, a combination of (ci) platinum and ruthenium, supported on (cii) a zeolite support (carrier) member. For example, the transition metal (component (ci)) may also include other transition metals such as palladium (Pd); cobalt (Co); copper (Cu); Rhodium (Rh); Rhenium (Re); nickel (Ni); and mixtures thereof. Preferably the transition metal component consists only of platinum and ruthenium. The acidic zeolite carrier member (component (cii)) can be derived from any zeolite or mixture of zeolite includes, for example, faujasite zeolite (“FAU”): sodium Y zeolite (NaY) (A sodium ion exchanged Y zeolite); mordenite zeolite (“MOR”); beta zeolite (“BEA”); chabazite zeolite (“CHA”); ferrierite zeolite (“FER”); Mobil-five zeolite (“MFI”) and mixtures thereof. These zeolites can preferably have an Si to Al ratio in the range of from 2 to 250. In some preferred embodiments, the heterogeneous catalyst useful in the present invention can be Pt—Ru supported on MOR. One or more additional catalysts such as Pd supported on a FAU support; Pt supported on a FAU, MOR, FER or CHA support; Rh supported on an FAU support, and mixtures thereof may also be used in addition to the Pt—Ru catalysts.

The heterogeneous catalyst of the present invention exhibits some advantageous properties. For example, the heterogeneous catalysts useful in the present invention provide a synergistic effect between the bimetallic compound of the catalyst and the zeolite carriers of the catalyst in order to catalyze direct ester hydrogenation. Otherwise, ether selectivities may decrease.

The heterogeneous catalyst, component (c), includes, for example, from 0.01 wt % to 20 wt % of the metallic compound based on the total weight of the heterogeneous catalyst in one embodiment, from 0.1 wt % to 10 wt % of the total metallic compound based on the total weight of the heterogeneous catalyst in another embodiment, and from 1 wt % to 5 wt % of the total metallic compound based on the total weight of the heterogeneous catalyst in still another embodiment. Preferably, the molar ratio of platinum to ruthenium is from 0.1 to 10, more preferably from 0.5 to 5, with rations of about 1:1 (such as (0.9 to 1.1) being most preferred.

The process equipment used to carry out the reduction process can be any conventional reactor such as a packed-bed reactor or a trickle bed reactor. The ester conversion and ether selectivities can be controlled via the reactor pressure, temperature, and surface residence time, as is generally understood in the art.

For example, the pressure of the process of the present invention is from 0.1 MPa to 10 MPa in one embodiment, from 2 MPa to 6 MPa in another embodiment, and from 6 MPa to 10 MPa in still another embodiment. Below the aforementioned pressure range may lead to lower reactivities or lower ether selectivities than disclosed herein. A pressure higher than the aforementioned pressure range may be sufficient to use in the present invention; however, it may require a higher cost in reactor construction and operation.

For example, the temperature of the process of the present invention is from 350 K to 650 K in one embodiment, from 400 K to 500 K in another embodiment, and from 500 K to 650 K in still another embodiment. Below the aforementioned temperature range may lead to lower reactivities than disclosed herein. A temperature higher than the aforementioned temperature range may lead to unwanted alkane and alcohol by-products; and therefore, in such a case the selectivities of the ether may decrease.

For example, the ester conversion of the process of the present invention is from 1% to 100% in one embodiment, from 1% to 50% in another embodiment, and from 50% to 100% in still another embodiment. In some embodiments, ester conversions higher than the aforementioned conversion range may lead to more side reaction products.

The process of the present invention may be carried out as a batch process or a continuous process. When using a batch process, in some embodiments, the residence time of the process of the present invention is, for example, from 0.1 hour (hr) to 24 hr in one embodiment, from 0.1 hr to 8 hr in another embodiment, and from 1 hr to 24 hr in still another embodiment. In some embodiments, residence times below the aforementioned residence time range may lead to a lower ester conversion; and in some embodiments, residence times above the aforementioned residence time range may lead to unwanted side reaction products.

When using a continuous process, in some embodiments, the residence time of the process of the present invention is, for example, from 0.1 second(s) to 100 s in one embodiment, from 1 s to 10 s in another embodiment, and from 10 s to 100 s in still another embodiment. Residence times below the aforementioned residence time range may lead to a lower ester conversion; and in some embodiments, residence times above the aforementioned residence time range may lead to unwanted side reaction products.

Some advantageous properties and/or benefits of using the reduction process of the present invention include, for example, the process of the present invention can achieve steady-state rates; and the process can provide better selectivities of product for competing reaction pathways for an ester compound, even better than other heterogeneous catalysts that do no feature zeolite supports. Also, conventional processes for producing an ether also produces salt whereas the process of the present invention does not generate salt.

After an ester compound undergoes the reduction process, the resulting ether product is formed. The turnover rate of ester to the ether product can be from 10⁻⁸ moles of ether per gram catalyst per second (mol/g_{cat}·S) to 10⁻⁵ mol/g_{cat}·s in one general embodiment, from 5×10⁻⁸ mol/g_{cat}·s to 5×10⁻⁶ mol/g_{cat}·s. Ester turnover rates below the aforementioned rate range may lead to a lower ether production rate; and in some embodiments, ester turnover rate above the aforementioned residence time range may lead to unwanted side reaction products.

The selectivity of the ether product can depend on whether a vapor process or liquid process is used to form the ether and whether a batch process or continuous process is used. In general, the selectivity of the direct ether product is >10% in one embodiment, from 10% to 25% in another embodiment, and from 25% to 60% in still another embodiment.

While the ether product produced by the process of the present invention can be a symmetric ether or an unsymmetric ether, as an illustration of the present invention and not to be limited thereby, the present invention process is described with reference to an unsymmetric ether. It has been surprisingly discovered that the process of the present invention is selective for unsymmetric ether because in the present invention process the ester is directly converted to ether, without undergoing ester hydrogenolysis and alcohol dehydration. Ester hydrogenolysis and alcohol dehydration are two processes that are known to not be selective for a specific ether.

If an unsymmetric ether is desired for a specific process or end use, then use of the present invention process is more advantageous than conventional processes because:

(1) An ether product is more stable than the corresponding ester product under basic and acid conditions. Also, the ether products of the present invention do not typically undergo hydrolysis which can occur at high humidity and/or high temperatures.

(2) It is believed that the hydrogen reaction chemistry of the present invention process maintains the backbone of the ester product intact. During the hydrogenation, only the oxygen molecule is broken away from the backbone which leaves the carbon molecules and backbone oxygen intact. In conventional reaction processes, the reaction breaks the backbone and combines parts back together under different reaction conditions. Thus, no direct hydrogenation/reduction of the ester to an ether occurs.

(3) The process of the present invention, minimizes undesirable side reactions that may detrimentally affect the selectivities of the desired ether product.

The ether product of the present invention has a minimal impact on the environment, since the ether product is derived from organic and renewable sources. For example, the ether product advantageously can be used as a global green and bio-based solvent to address the stringent regulations imposed on chemical-based industrial solvents in relation to toxicity, non-biodegradability, volatile organic compound (VOC) emissions, and the like. Green and bio-based solvents are typically used in paints and coatings applications. Other applications include adhesives, pharmaceuticals, and printing inks. In some embodiments, the ether product can be used as a foam control agent and a flavor additive. In other embodiments, the ether product can be used in cosmetics and personal care applications.

The present invention provides biobased solvents at a cost and performance advantage to known solvents in the industry. In addition, the chemical transformation provided by the present invention process could be useful to produce, for example, bio-based surfactants, defoamers and lubricants with both an economically and environmentally favorable process.

The ether generation process of the present invention can also be used to develop: (1) a more robust capping process to overcome the issues of limited reactant alkyl chloride types and final product impurities; (2) new capped low viscosity-low volatility lubricants; and (3) new surfactants and new biobased defoamers for food and pharmacy applications, metalworking fluids applications, and other applications utilizing an ether solvent.

### EXAMPLES

The following Inventive Examples (Inv. Ex.) and Comparative Examples (Comp. Ex.) (collectively, “the Examples”) are presented herein to further illustrate the present invention in detail but are not to be construed as limiting the scope of the claims. Unless otherwise stated all parts and percentages are by weight.

### Catalysts

The catalyst (“Cat.”) formulations used in the Examples are described in Table 1 for inventive examples) and Table 2 (for comparative examples).

TABLE 1

| | | | | | Target | | | |
|---|---|---|---|---|---|---|---|---|
| | | | | Support | % mol | Molar | | |
| Catalyst | Metal | Metal | | Si/Al | total | ratio | | |
| No. | 1 | 2 | Support | Ratio | metal | M2/M1 | M1/Al | Preparation method |
| Cat. 1 | Pt | Ru | MOR | 10 | 0.32 | 1.0 | 0.1 | IWI, co-impregnation |
| Cat. 2 | Pt | Ru | MOR | 120 | 0.32 | 1.0 | 0.6 | IWI, co-impregnation |
| Cat. 3 | Pt | Ru | MOR | 120 | 0.32 | 10.0 | 0.1 | IWI, co-impregnation |
| Cat. 4 | Pt | Ru | MOR | 120 | 0.32 | 0.5 | 0.8 | IWI, co-impregnation |

TABLE 2

| | | | | | Target | | | |
|---|---|---|---|---|---|---|---|---|
| | | | | Support | % mol | Molar | | |
| Catalyst | Metal | Metal | | Si/Al | total | ratio | | |
| No. | 1 | 2 | Support | Ratio | metal | M2/M1 | M1/Al | Preparation method |
| Cat. 5 | Pt | N/A | MOR | 10 | 0.32 | N/A | 0.4 | IWI |
| Cat. 6 | Ru | N/A | MOR | 10 | 0.61 | N/A | | IWI |
| Cat. 7 | Pt | N/A | MOR | 120 | 0.32 | N/A | 1.2 | IWI |
| Cat. 8 | Pt | Pd | MOR | 120 | 0.32 | 1.0 | 0.6 | IWI, co-impregnation |
| Cat. 9 | Pt | Co | MOR | 120 | 0.32 | 10.0 | 0.1 | IWI, co-impregnation |
| Cat. 10 | Pt | Ni | MOR | 120 | 0.32 | 1.0 | 0.6 | IWI, co-impregnation |
| Cat. 11 | Pt | Mo | MOR | 120 | 0.32 | 1.0 | 0.6 | IWI, co-impregnation |
| Cat. 12 | Pt | Mo | ZrO₂ | N/A | 2.4 | 5.3 | N/A | Rotary evaporation |
| Cat. 13 | Pt | Mo | ZrO₂ | N/A | 2.4 | 5.3 | N/A | IWI, co-impregnation |

The indicated transition metal particles (Pt, Ru, Pd, Co, and/or Ni,) are deposited onto the indicated support (MOR zeolite or ZrO₂,) using incipient wetness impregnation (IWI), or rotary evaporation method, as indicated.

The “IWI” and “IWI, co-impregnation” method involves impregnating the indicated support with an aqueous solution of precursor(s) of the indicated transition metals as follows: The precursors used for Pt, Ru, Pd, Co, Ni and Mo are Pt(NH₃)₄(NO₃)₂, Ru(NO)(NO₃)₃, Pd(NH₃)₄(NO₃)₂, Co(NO₃)₂·6H₂O, Ni(NO₃)₂·6H₂O, and (NH₄)₆Mo₇O₂₄, respectively. The MOR zeolites are commercially available from ACS Materials (Si/Al=10), and from Tosoh (Si/Al=120). The IWI method involves preparing an aqueous solution with the precursor concentration adjusted to the indicated weight loading. An equivalent volume of the support pore volume was added dropwise to the support achieving incipient wetness. The impregnated supports are then dried in a static oven at 353 K for more than 12 h, and then calcinated at 773 K for 2 h in air (100 cm³ min⁻¹). Finally, the sample was reduced in flowing 20% H₂/He at 423 K for 2 h (5K min⁻¹).

The “rotary evaporation” method is as published in JACS Au, 2022, 2 (3), 665-672. An aqueous solution of (NH₄)₆Mo₇O₂₄ (37.5 mM) and ZrO₂ (15 g) is added to distilled water (500 mL) at room temperature. The water is removed by rotary evaporation under reduced pressure to obtain the solid product, and the resulting sample is dried at 343 K overnight. The dried powder and an aqueous solution of H₂PtCl₆ (100 mM) are added to distilled water (500 mL). Then, rotary evaporation method is introduced again to remove water. The powders are dried at 343 K overnight. The sample is then calcined in flowing air (200 cm³ min⁻¹) at 773 K for 3 h (5K min⁻¹) and then, reduced in flowing 20% H₂/He at 423 K for 2 h (5K min⁻¹).

### Liquid Phase Reaction

Catalysts 1-13 are tested in a liquid-phase reactor. The liquid-phase reactor is a trickle bed reactor comprised of stainless-steel tube (⅜″ O.D.) containing 1-6 g of catalyst (30-60 mesh), which is held at the center of the reactor using pyrex glass rods and packed glass wool. The reactor is heated with an aluminum clamshell, including two heat cartridges that are controlled by an electronic temperature controller. The reaction temperature is measured by a K-type thermocouple contained within a ⅛″ stainless steel sheath that is coaxially aligned within the reactor and submerged within the aluminum clamshell. The system is pressurized up to 6.6 MPa using a dome-loaded back pressure regulator which is controlled by an electronic pressure regulator. The reactor pressure is monitored using a digital pressure gauge. H₂ (Ultra High Purity) and He (Ultra High Purity) gas flow rates are controlled using mass flow controllers. Neat propyl acetate (PA) is dried using molecular sieve 3A beads (1-2 mm) prior to injection of the reactant into the reactor. The flow rate of dried PA in the liquid phase (C₅H₁₀O₂, 99.5%) is controlled using an HPLC pump fed through a stainless-steel tube ( 1/16″ outer O.D., 0.006″ I.D.) within a cross-flow of H₂ and He. Catalysts are pretreated in situ by heating to 503 K at 5 K·min⁻¹ and held for 1 h in flowing a mixture (50 cm³·min⁻¹) of H₂ (20 kPa, Ultra High Purity) and He (81 kPa, Ultra High Purity) prior to the measurements. The effluent of the reactor is passed through a stainless cooling chamber including cold water (˜283 K), and then gas and liquid products are split into a gas stream and a liquid stream using a stainless-steel gas-liquid separator. The liquid products collected in the gas-liquid separator are delivered using an automatic product delivery system, consisting of HPLC pump and wet/wet differential pressure transducer (0-10 inH₂O), to high pressure liquid sampling valve (LSV, 1 μL of injection volume) which is attached to online gas chromatography. At the outlet of LSV, manual BPR is installed so that the pressure of liquid pressure was maintained at 1.4 MPa to prevent the products from evaporation in the LSV sampling system. The gas and liquid products are characterized using online gas chromatography. Gas sampling valve and LSV were used to inject gas and liquid products into split/splitless inlets, respectively. The gas chromatograph (GC) is equipped with two capillary columns (GS-GASPRO 60 m length, 0.32 mm inner diameter for gas products and DB-Wax UI, 60 m length, 0.25 mm inner diameter, 0.25 μm for liquid products) connected to two flame ionization detectors (FID) to quantify the concentrations of species. Sensitivity factors for gas and liquid products are determined using gaseous standards and methanizer respectively. Control of the reaction pressure and temperature, reactant and product flow rates, and the GC samplings are automated to allow for continuous measurements. Conversions are calculated on a carbon basis based on the amount of carbon that appears in the products. The carbon balance closes within +10%. Catalyst deactivation is corrected by linear interpolation of time-on-stream data.

The reactions for Inv. Ex. 1-4 and Comp. Ex. A-G are carried out at 383 K under a H₂ pressure of 6431 kPa, and at an ester pressure of 119 kPa. The reactions for Comp. Ex. H—I are carried out at 373 K under a H₂ pressure of 6462 kPa, and at an ester pressure of 88 kPa, a similar condition as published in JACS Au, 2022, 2 (3), 665-672. In liquid-phase reactor, the ester is maintained in liquid state due to its high concentration in feed mixture under reaction pressures and temperatures. Results of the liquid phase experiments are shown in Table 3

TABLE 3

| | | | | | | | | Abso- | | | |
|---|---|---|---|---|---|---|---|---|---|---|---|
| | | | | | | | | lute | Abso- | Abso- | |
| | | | | | | | | Selec- | lute | lute | |
| | | | | | | | Hydro- | tivity | Selec- | Selec- | Direct |
| | | | | | Feed Rate | | genation | (% | tivity | tivity | Ether |
| | Cata- | | H₂ | C₅H₁₀O₂ | (mol | | Rate (mol | Ethyl | (% | (% | Selec- |
| Ex- | lyst | Temp. | Press. | Press. | C₅H₁₀O₂) · | Conv. | C₅H₁₀O₂) · | Propyl | Diethyl | Dipropyl | tivity |
| ample | No. | (K) | (kPa) | (kPa) | (g_{cat} · s)⁻¹ | (%) | (g_{cat} · s)⁻¹ | Ether) | Ether) | Ether) | (%) |
| Inv. | Cat. | 383 | 6431 | 119 | 2.12 × 10⁻⁵ | 5.0 | 2.77 × 10⁻⁷ | 25.8 | 0.3 | 2.2 | 91.2 |
| Ex. 1 | 1 | | | | | | | | | | |
| Inv. | Cat. | 383 | 6431 | 119 | 4.04 × 10⁻⁵ | 8.6 | 8.83 × 10⁻⁷ | 25.4 | 0.4 | 0.0 | 98.4 |
| Ex. 2 | 2 | 383 | 6431 | 119 | 2.89 × 10⁻⁵ | 11.2 | 7.63 × 10⁻⁷ | 23.6 | 0.4 | 0.0 | 98.3 |
| | | 383 | 6431 | 119 | 1.73 × 10⁻⁵ | 16 | 5.88 × 10⁻⁷ | 21.2 | 0.4 | 0.0 | 98.1 |
| Inv. | Cat. | 383 | 6431 | 119 | 1.92 × 10⁻⁵ | 8.6 | 4.03 × 10⁻⁷ | 24.3 | 0.8 | 0.1 | 96.4 |
| Ex. 3 | 3 | 383 | 6431 | 119 | 4.49 × 10⁻⁵ | 4.1 | 5.47 × 10⁻⁷ | 29.7 | 0.8 | 0.1 | 97.1 |
| | | 383 | 6431 | 119 | 6.41 × 10⁻⁵ | 2.8 | 5.86 × 10⁻⁷ | 32.3 | 0.9 | 0.1 | 97.0 |
| Inv. | Cat. | 383 | 6431 | 119 | 2.12 × 10⁻⁵ | 7.1 | 3.55 × 10⁻⁷ | 23.7 | 0.1 | 2.2 | 91.2 |
| Ex. 4 | 4 | | | | | | | | | | |
| Comp. | Cat. | 383 | 6431 | 119 | 2.35 × 10⁻⁶ | 0.4 | 2.53 × 10⁻⁸ | 27.4 | 6.7 | 0.9 | 78.3 |
| Ex.A | 5 | | | | | | | | | | |
| Comp. | Cat. | 383 | 6431 | 119 | 1.84 × 10⁻⁵ | 2.0 | 5.53 × 10⁻⁹ | 1.5 | 0.0 | 10.1 | 12.9 |
| Ex.B | 6 | | | | | | | | | | |
| Comp. | Cat. | 383 | 6431 | 119 | 2.96 × 10⁻⁵ | 0.8 | 1.12 × 10⁻⁷ | 48.9 | 2.2 | 0.3 | 95.1 |
| Ex.C | 7 | | | | | | | | | | |
| Comp. | Cat. | 383 | 6431 | 119 | 2.16 × 10⁻⁵ | 0.4 | 3.77 × 10⁻⁸ | 39.7 | 17.3 | 0.7 | 68.8 |
| Ex.D | 8 | | | | | | | | | | |
| Comp. | Cat. | 383 | 6431 | 119 | 6.61 × 10⁻⁶ | 0.3 | 4.42 × 10⁻¹⁰ | 2.5 | 48.8 | 0.4 | 4.8 |
| Ex.E | 9 | | | | | | | | | | |
| Comp. | Cat. | 383 | 6431 | 119 | 1.92 × 10⁻⁵ | 0.3 | 1.50 × 10⁻⁹ | 3.5 | 31.6 | 0.0 | 10.0 |
| Ex.F | 10 | | | | | | | | | | |
| Comp. | Cat. | 383 | 6431 | 119 | 2.26 × 10⁻⁵ | 0.2 | 8.94 × 10⁻⁹ | 12.4 | 13.5 | 0.0 | 47.9 |
| Ex.G | 11 | | | | | | | | | | |
| Comp. | Cat. | 373 | 6462 | 88 | 9.33 × 10⁻⁶ | 0.2 | 2.76 × 10⁻¹⁰ | 1.6 | 0.0 | 0.0 | 100.0 |
| Ex.H | 12 | | | | | | | | | | |
| Comp. | Cat. | 373 | 6462 | 88 | 1.87 × 10⁻⁵ | 0.2 | 3.30 × 10⁻⁹ | 11.1 | 0.6 | 0.0 | 94.9 |
| Ex.l | 13 | | | | | | | | | | |

In Inv. Ex. 1-4, the selectivities for formation of dipropyl ether and diethyl ether from propyl acetate are, in most cases, lower than 3%, while the relative selectivity for ethyl propyl ether among all the three ether products in most cases is higher than 90%. Such performance indicates that substantially all of the ethyl propyl ether is formed based on a direct hydrogenation reaction pathway of propyl acetate, since the alcohol dehydration has no preference in selectivities for symmetric or unsymmetric ether. Notably, the absolute ethyl propyl ether selectivities for Inv. Ex. 1-4 are all above 20%. Such results significantly surpass the prior art performance on transition metal/metal oxide catalysts, for example U.S. Patent application 63/107,739, which claimed >5% in absolute ethyl propyl ether selectivity and >85% direct ether selectivity.

Comparing the results of Inv. Ex. 1 and Inv. Ex. 2, the Pt—Ru on MOR with 120 Si/Al ratio (Cat. 2) shows better performance to Pt—Ru on MOR with 10 Si/Al (Cat. 1). The hydrogenation rate of Cat. 2 is 8.83×10⁻⁷ (mol C₅H₁₀O₂)·(g_{cat}·s)⁻¹, over 3 times higher than Cat. 1, which is 2.77×10⁻⁷ (mol C₅H₁₀O₂)·(g_{cat}·s)⁻¹, under the same reaction condition. Moreover, Cat. 2 has higher direct ether selectivity at 98.4% than Cat. 1 at 91.2%.

Comparing the results of Inv. Ex. 2, Inv. Ex. 3 and Inv. Ex. 4, all the Pt—Ru with different Pt to Ru ratios (Cat. 2, Cat. 3, Cat. 4), while maintaining total metal moles the same, show good hydrogenation rates, absolute ether selectivities and relative ether selectivities. The best hydrogenation rate is obtained from Cat. 2 with a Pt to Ru ratio of 1.

The results of Comp. Ex. A-C are obtained from monometallic Pt or Ru MOR zeolite catalysts (Cat. 5, Cat. 6 and Cat. 7). The purpose of providing these comparative examples is to demonstrate the superior performance of bimetallic Pt—Ru MOR catalysts to their corresponding monometallic MOR catalysts. Comparing Inv. Ex. 1 and Comp. Ex. A, Pt—Ru-MOR10 catalyst (Cat. 1) shows over 10 times higher hydrogenation rate than Pt-MOR10 (Cat. 5), 2.77×10⁻⁷ (mol C₅H₁₀O₂)·(g_{cat}·s)⁻¹ V.S. 2.53×10⁻⁸ (mol C₅H₁₀O₂)·(g_{cat}·s)⁻¹. Meanwhile, the absolute ether selectivity from Pt—Ru-MOR10 is comparable to Pt-MOR10, which is 25.8% to 27.4%. The Ru-MOR10 (Cat. 6), in contrast, shows both poor hydrogenation rate (5.53×10⁻⁹ (mol C₅H₁₀O₂)·(g_{cat}·s)⁻¹), and absolute ether selectivity (1.5%). These results indicate that bimetallic Pt—Ru-MOR has better performance for ester reduction to ether comparing to either Pt-MOR or Ru-MOR monometallic catalysts. Similar conclusion can also be drawn from the comparison of Inv. Ex. 2 and Comp. Ex. C, as the bimetallic Pt—Ru-MOR 120 (Cat.2) has a hydrogenation rate of 8.83×10⁻⁷ (mol C₅H₁₀O₂)·(g_{cat}·s)⁻¹, much higher than monometallic Pt-MOR 120 (Cat. 7) with a hydrogenation rate of 1.12×10⁻⁷ (mol C₅H₁₀O₂)·(g_{cat}·S)⁻¹, by a factor of 7. However, the Pt-MOR120 shows better absolute ether selectivity (48.9%) than Pt—Ru-MOR120 (25.4%). In general, the results indicate that bimetallic Pt—Ru zeolite catalyst has significant enhancement on hydrogenation rate, surpassing the prior art performance of monometallic zeolite catalyst by 5˜10 times (see U.S. patent application 63/276,311).

The results of Comp. Ex. D-Comp. Ex. G (Cat. 8-Cat. 11) indicate that not all Pt-M bimetallic MOR zeolites show enhanced performance for ester reduction to ethers, either on hydrogenation rate or ether selectivity. Pt—Pd-MOR120 (Cat. 8) shows high absolute ether selectivity up to 39.7%, however, it also forms symmetrical ether products, giving the low relative ether selectivity of 68.8%. Pt—Co-MOR120 (Cat. 9), Pt—Ni-MOR120 (Cat. 10) and Pt—Mo-MOR120 (Cat. 11) all show low absolute ether selectivities below 15%, indicating that those catalysts are less effective to reduce esters to ethers.

The results of Comp. Ex. F and Comp. Ex. G are obtained from the prior art bimetallic catalyst (JACS Au, 2022, 2 (3), 665-672), which is relevant to current invention. The Cat. 12 Pt—Mo/ZrO₂ catalyst is a reproduced catalyst using the method described by prior art. An IWI version of Pt—Mo/ZrO₂ catalyst (Cat. 13) is also prepared and tested under prior art reaction condition. First, Pt—Mo/ZrO₂ catalysts shows low absolute desired ether selectivities, 1.6% from Cat. 12 and 11.1% from Cat. 13. The products generated from Pt—Mo/ZrO₂ catalysts include significant amount of byproducts such as alcohols, and light alkanes. Second, the hydrogenation rates of Cat. 12 and Cat. 13 are as low as 2.76×10⁻¹⁰ (mol C₅H₁₀O₂)·(g_{cat}·s)⁻¹ and 3.30×10⁻⁹ (mol C₅H₁₀O₂)·(g_{cat}·s)⁻¹. The Pt—Ru-MOR bimetallic catalysts disclosed in this invention have higher hydrogenation rates by over two orders of magnitude in general, compared to these prior art catalysts.

### Gas Phase Reaction

Catalysts 2 and 7, are also tested in a vapor-phase reactor. The vapor-phase reactor is a trickle bed reactor. Rate and selectivity measurements are carried out in a packed bed reactor held within a stainless-steel tube (⅜″ O.D.) containing ˜10-200 mg of catalyst, which is held at the center of the reactor using glass rods and packed glass wool. The tubular reactor is placed within a three-zone furnace that is controlled by an electronic temperature controller. The catalyst temperature is measured by a K-type thermocouple contained within a 1/16″ stainless steel sheath that is coaxially aligned within the reactor and submerged within the catalyst bed. The volume of the catalyst bed is kept constant at 1.4 cm³ of material by mixing excess SiC (Carborex green 36) with the desired amount of catalyst. The system is pressurized using a back pressure regulator, which is controlled by an electronic pressure regulator (“EPR”). The reactor pressure is monitored upstream and downstream of the catalyst bed using a digital pressure gauge and the EPR, respectively.

Hydrogen (H₂, Ultra High Purity 5.0) and Helium (He, Ultra High Purity 5.0) gas flow rates are controlled using mass flow controllers. The flow rates of liquid propyl acetate (C₅H₁₀O₂, ≥99.5%) is controlled using a stainless-steel syringe pump with a Hastelloy cylinder fed through a PEEK tube ( 1/16″ outer O.D., 0.01″ I.D.) the exit of which is positioned within a small bed of non-porous sand (SiO₂ 50-70 mesh particle size) within a cross flow of H₂. The transfer lines surrounding the liquid inlet are kept at 373 K using heating tape to avoid condensation. All transfer lines downstream of the liquid inlet are heated above 373 K using heating tape and line temperatures are monitored with K-type thermocouples displayed on a digital reader.

Catalysts are pretreated in situ by heating to the desired temperature at 0.05 K s⁻¹ and holding for the desired time within 101 kPa flowing hydrogen (H₂, Ultra High Purity 5.0) at 100 cm³ min⁻¹ prior to all catalytic measurements. The effluent of the reactor is characterized using on-line gas chromatography. The gas chromatograph (GC) is equipped with a capillary column (DB-624 UI, 30 m length, 0.25 mm inner diameter, 1.40 μm) connected to a flame ionization detector to quantify the concentrations of combustible species. Sensitivity factors and retention times for all components are determined using gaseous and liquid standards. Control of the reactor pressure and temperature, reactant flowrates, and the GC sampling are automated to allow for continuous measurements. Conversions are calculated on a carbon basis based on the amount of carbon that appears in the products. The carbon and oxygen balance closes within +20%. Reactor conditions during rate and selectivity measurements are varied by sequentially decreasing and then increasing the reactant pressure over the full range such that one or more of the conditions is measured at least twice throughout the experiment to ensure that measured trends are not a result of systematic deactivation.

In Inv. Ex. 5 and Comp. Ex. J. the hydrogenation is carried out in a vapor-phase reactor using the indicated catalysts. The reactions for Inv. Ex. 5 and Comp. Ex. J are carried out at 356-383 K under a H₂ pressure of 6431 kPa, and at an ester pressure of 10 kPa. In vapor-phase reactor, the ester is maintained in vapor state due to its lower concentration in feed mixture under reaction pressures and temperatures, compared to liquid-phase reactor. The ether product selectivities are described in Table 4.

TABLE

| | | | | | | | | Abso- | | | |
|---|---|---|---|---|---|---|---|---|---|---|---|
| | | | | | | | | lute | Abso- | Abso- | |
| | | | | | | | | Selec- | lute | lute | |
| | | | | | | | Hydro- | tivity | Selec- | Selec- | Direct |
| | | | | | Feed Rate | | genation | (% | tivity | tivity | Ether |
| | Cata- | | H₂ | C₅H₁₀O₂ | (mol | | Rate (mol | Ethyl | (% | (% | Selec- |
| Ex- | lyst | Temp. | Press. | Press. | C₅H₁₀O₂) · | Conv. | C₅H₁₀O₂) · | Propyl | Diethyl | Dipropyl | tivity |
| ample | No. | (K) | (kPa) | (kPa) | (g_{cat} · s)⁻¹ | (%) | (g_{cat} · s)⁻¹ | Ether) | Ether) | Ether) | (%) |
| Inv. | Cat. | 383 | 6431 | 10 | 1.40 × 10⁻⁵ | 19.9 | 1.17 × 10⁻⁶ | 39.9 | 0.6 | 3.9 | 89.7 |
| Ex. 5 | 2 | 370 | 6431 | 10 | 1.40 × 10⁻⁵ | 14.9 | 8.00 × 10⁻⁷ | 37.5 | 0.4 | 4.2 | 89.1 |
| | | 356 | 6431 | 10 | 1.40 × 10⁻⁵ | 10.0 | 5.20 × 10⁻⁷ | 34.7 | 0.4 | 4.5 | 87.6 |
| Comp. | Cat. | 383 | 6431 | 10 | 2.47 × 10⁻⁶ | 17.3 | 2.03 × 10⁻⁷ | 47.7 | 3.2 | 4.6 | 85.9 |
| Ex.J | 7 | 370 | 6431 | 10 | 2.47 × 10⁻⁶ | 11.9 | 1.38 × 10⁻⁷ | 47.0 | 1.9 | 5.1 | 87.0 |
| | | 358 | 6431 | 10 | 2.47 × 10⁻⁶ | 7.7 | 8.74 × 10⁻⁸ | 46.1 | 1.2 | 5.8 | 86.8 |

The results of In Inv. Ex. 5, are obtained from Cat. 2 in a vapor-phase reactor. The results showed that the Pt—Ru-MOR120 is also effective in ester reduction to ether with high selectivities and rates. The absolute selectivities of desired ether range from 34.7% to 39.9% at temperatures varying from 356 to 383K, while maintaining high relative ether selectivies; above 85%. The hydrogenation rates are measured about 5.20×10⁻⁷ (mol C₅H₁₀O₂)·(g_{cat}·s)⁻¹ to 1.17×10⁻⁶ (mol C₅H₁₀O₂)·(g_{cat}·s)⁻¹.

The results of Comp. Ex. J are obtained from the corresponding monometallic Pt-MOR120 catalyst (Cat. 7), for comparison purpose with Pt—Ru-MOR120 (Cat. 2) under the same reaction conditions. The Pt-MOR120 shows good absolute ether selectivities from 46.1 to 47.7% under tested reaction temperature, with good relative ether selectivies above 85% as well. However, comparing the hydrogenation rates, the results from bimetallic Pt—Ru-MOR120 are over 5 times higher than monometallic Pt-MOR120, which are measured about 8.74×10⁻⁸ (mol C₅H₁₀O₂)·(g_{cat}·s)⁻¹ to 2.03×10⁻⁶ (mol C₅H₁₀O₂)·(g_{cat}·s)⁻¹.

What is claimed is:

1. A process for producing an ether product by direct selective reduction of an ester comprising treating (a) at least one ester with (b) hydrogen and in the presence of (c) a heterogeneous catalyst to directly and selectively reduce the at least one ester by hydrogenation to form the at least one ether; wherein the heterogeneous catalyst comprises platinum and ruthenium deposited on a zeolite carrier.

2. The process of claim 1, wherein the ester is an ester containing straight or branched-chain alkyl groups; cyclic or non-cyclic alkyl groups; and mixtures thereof.

3. The process of claim 1, wherein the molar ratio of platinum to ruthenium is in a range of from 0.1 to 10.

4. The process of claim 1 wherein the total amount of transition metal deposited onto the zeolite carrier is in an amount of from 0.01 to 20 percent by weight of the metallic compounds based on the total weight of the heterogeneous catalyst.

5. The process of claim 1, wherein the zeolite comprises at least one of FAU, sodium Y zeolite, MOR, BEA, CHA, FER, MFI and mixtures thereof.

6. The process of claim 1 where the zeolite has an Si to Al ratio in the range of from 1 to 300.

7. The process of claim 1, wherein the temperature of the process is from 350 K to 650 K; and wherein the pressure of the process is from 0.1 MPa to 10 MPa.

8. The process of claim 1, wherein the ether is an unsymmetric ether.

9. The process of claim 1, wherein the process is a vapor-phase reduction process carried out under vapor-phase process conditions.

10. The process of claim 1, wherein the process is a liquid-phase reduction process carried out under liquid-phase process conditions.

11. The process of claim 1 where the zeolite carrier is MOR.

12. A solvent comprising an ether produced from the process of claim 1.
